(19) United States
(12) Patent Application Publication
(10) Pub. No.: US 2017/0252447 A1
(43) Pub. Date: Sep. 7, 2017

(54) INFUSABLE SOLUTION FOR LOCAL TREATMENT OF BLOOD VESSELS AND VASCULAR GRAFTS AND METHODS OF USING SUCH A SOLUTION

(71) Applicant: Cylerus, Inc., Melbourne, FL (US)

(72) Inventors: Ronald J. Shebuski, Melbourne, FL (US); Stephen R. Hanson, Edmonds, WA (US); Christopher F. Toombs, Sammamish, WA (US)

(21) Appl. No.: 15/447,073

(22) Filed: Mar. 1, 2017

Related U.S. Application Data
(60) Provisional application No. 62/304,203, filed on Mar. 5, 2016.

(51) Int. Cl. A61K 47/26; A61K 9/00; A61K 31/436; A61K 47/10; A61K 9/08

(52) U.S. Cl. CPC A61K 47/26; A61K 47/10; A61K 9/08; A61K 31/436; A61K 9/0019; A61K 9/0004

(57) ABSTRACT

A novel treatment for dialysis patients needing a hemodialysis graft by providing a therapeutic formulation that can be infused to such patients, which may mitigate graft-related neointimal hyperplasia, thrombosis, and/or related mechanisms of graft failure. The therapeutic formulation may include sirolimus and/or other olimus drug(s). The therapeutic formulation may be infusible and stable in solution for such a pharmaceutical and therapeutic purpose. The therapeutic formulation may have a pharmaceutically acceptable shelf life such that the pharmaceutical agent remains chemically stable (e.g., does not precipitate) for a suitable number of days (e.g., at least 30 days, at least 60 days, at least 90 days, etc.) at 37° C. The therapeutic formulation may be suitable for application to a patient's blood vessel/vascular graft via an infusion pump. For instance, the therapeutic formulation may be suitable for application to the patient's hemodialysis graft location.

### CROSS-REFERENCE TO RELATED APPLICATION(S)

This application claims the benefit of U.S. Provisional Application No. 62/304,203, filed Mar. 5, 2016 and entitled “Infusable Solution for Local Treatment of Blood Vessels and Vascular Grafts and Methods of Using Such a Solution,” the entirety of which is incorporated by reference herein.

### STATEMENT REGARDING FEDERALLY SPONSORED RESEARCH AND DEVELOPMENT

This invention was made with government support under Grant Number 1R43HL106907-01A1 REVISED awarded by the National Heart, Lung, and Blood Institute of the National Institutes of Health. The government has certain rights in this invention.

### BACKGROUND

Dialysis is for people who have renal failure, otherwise known as kidney failure. Kidney failure means that the patient's kidneys are not able to cleanse the blood of wastes, including urea, and extra fluid and the patient is unable to micturate. Thus, in order to cleanse the blood of waste, dialysis is traditionally employed. Dialysis takes about three hours and is typically performed 2-4 times per week for patients experiencing kidney failure.

There are two types of dialysis for people with kidney failure: (1) hemodialysis, where the blood is withdrawn from the body into a machine (dialyzer) that uses a special semi-permeable membrane to filter wastes and remove extra fluid from the blood, and also restore the electrolyte balance in the blood; and (2) peritoneal dialysis, where a fluid is placed into the abdominal cavity through a special tube called a catheter and is left in place for several hours, after which it is removed. This fluid removes wastes and extra fluid from the body.

Other than dialysis, kidney transplantation typically is the only other option for people with kidney failure. Due to a shortage of donor kidneys, a majority of people with kidney failure are on dialysis.

One relatively desirable form of hemodialysis vascular access is called a fistula. To make a fistula, a surgeon connects an artery to a vein in the forearm or upper arm of the patient. With time, usually one to three months, the vein enlarges and becomes ready to receive the large needles used to withdraw and replace blood during dialysis. A fistula can last for many years if the vein enlarges and the fistula “develops.” Only about three-quarters of fistulas develop or mature. During the time that a fistula is developing, if hemodialysis is necessary, another form of vascular access will be employed, usually a catheter.

In some patients, the arteries and/or veins are not suitable for making a fistula or these patients may have had a prior fistula that failed. In these patients, a shunt or graft can be used as an alternative form of dialysis access. A graft is a piece of synthetic tubing that is inserted by a surgeon and connects the artery to the vein. Unlike fistulas, grafts do not need to “develop” and are ready for use in most instances within four weeks after placement. A disadvantage of grafts is that they do not last as long as fistulas and can develop obstructive narrowing due to tissue ingrowth (neointimal hyperplasia) and clotting (thrombosis) within the blood vessel more frequently. In addition, grafts can get infected—something which does not happen very often with fistulas.

Hemodialysis access failure due to neointimal hyperplasia and thrombosis is a relatively common cause of morbidity for patients on hemodialysis. A substantial percentage of hospitalizations of these patients are due to vascular access complications. Results appear to be worsening since, in practice, the interval between access placement and the need for a procedure to restore access patency has been shortening. In addition, expenditures for reconstituting patency are substantial and increasing.

Vascular access neointimal hyperplasia and thrombosis are major problems for hemodialysis patients. A 7.75 year study using intra-access venous pressure monitoring at zero dialyzer blood flow found that in a group of 832 patients, the percentage of prosthetic bridge grafts increased from 65% to 80% (Besarb et al., Utility of intra-access pressure monitoring in detecting and correcting venous outlet stenoses prior to thrombosis, Kidney Int. 1995 May; 47(5):1364-73).

Another study found extensive morbidity related to hemodialysis vascular access exists among end-stage renal disease (ESRD) patients, and studying Medicare ESRD patient data from 1984, 1985, and 1986 found that 15 to 16% of hospital stays among prevalent ESRD patients were associated with vascular access-related morbidity (Feldman et al., Hemodialysis vascular access morbidity in the United States, Kidney Int. 1993 May; 43(5): 1091-6).

During 1981, 946 patients with advanced renal failure who were maintained by dialysis were studied in another study to assess the frequency and the duration of hospitalizations and to identify complications that prompted hospitalization (Carlson et al., Hospitalization in Dialysis Patients, Mayo Clin Proc. 1984 November; 59(11):769-75). 558 patients (59%) were hospitalized a total of 1,207 times and the major reasons for hospitalization were dialysis access problems (25%), gastrointestinal complications (13%), and cardiac complications (13%).

In another study, new data on the magnitude and growth of vascular access-related hospitalization in the United States was presented, demonstrating that the costs of this morbidity will soon exceed $1 billion per year. (Feldman et al., Hemodialysis vascular access morbidity, J Am Soc Nephrol. 1996 April; 7(4):523-35). This study also demonstrated the continuing evolution of medical practice away from the use of arteriovenous fistulae in favor of more reliance on synthetic bridge grafts. The study concludes that “[t]o reduce vascular access-related morbidity, strategies must be developed . . . to prevent and detect appropriately early synthetic vascular access dysfunction.” Feldman, et al., at Abstract.

### BRIEF DESCRIPTION OF THE DRAWINGS/FIGURES

The accompanying drawings, which are incorporated herein and form part of the specification, illustrate embodiments of the present invention and, together with the description, further serve to explain the principles involved and to enable a person skilled in the relevant art(s) to make and use the disclosed technologies.

FIG. 1 depicts a graph showing stability of three example formulations of sirolimus in accordance with an embodiment.

FIG. 2 depicts a graph showing whole blood levels of sirolimus in pigs infused intravenously with stable sirolimus formulations in accordance with an embodiment.

FIG. 3 depicts a table that shows detailed results of a stability study in accordance with an embodiment.

FIG. 4 depicts a graph that shows summary results of the stability study in accordance with an embodiment.

FIG. 5 depicts a flowchart of an example method for using an infusible solution in accordance with an embodiment.

The features and advantages of the disclosed technologies will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### I. Introduction

The following detailed description refers to the accompanying drawings that illustrate exemplary embodiments of the present invention. However, the scope of the present invention is not limited to these embodiments, but is instead defined by the appended claims. Thus, embodiments beyond those shown in the accompanying drawings, such as modified versions of the illustrated embodiments, may nevertheless be encompassed by the present invention.

References in the specification to “one embodiment,” “an embodiment,” “an example embodiment,” or the like, indicate that the embodiment described may include a particular feature, structure, or characteristic, but every embodiment may not necessarily include the particular feature, structure, or characteristic. Moreover, such phrases are not necessarily referring to the same embodiment. Furthermore, when a particular feature, structure, or characteristic is described in connection with an embodiment, it is submitted that it is within the knowledge of one skilled in the relevant art(s) to implement such feature, structure, or characteristic in connection with other embodiments whether or not explicitly described.

Example embodiments are described herein with reference to a drug agent “sirolimus” for illustrative purposes. Sirolimus is included in a therapeutic class of “limus” or “olimus” drugs, including but not limited to everolimus, zotarolimus, tacrolimus, and biolimus. The example embodiments described herein are applicable to the entire class of olimus agents, and the term “sirolimus” is used merely as one example of an agent in the olimus/limus class. It will be recognized by persons skilled in the art that descriptions herein with regard to sirolimus are applicable to all olimus/limus agents. It will be further recognized that one skilled in the art may employ appropriate changes applicable to each therapeutic agent without departing from the scope of the invention.

### II. Example Embodiments

A stent is a relatively small stainless steel tube that may be permanently placed inside an artery to keep the artery open. Implantation of a stent is fairly common in cardiovascular therapies, even though such implantation may cause stent restenosis. Stent restenosis is a neointimal proliferation that may necessitate repeated revascularization. Although using drug-eluting stents, such as a sirolimus eluting stent, may reduce the occurrence of stent restenosis, using such stents may increase the risk of stent thrombosis. Stent thrombosis is an occlusion (e.g., sudden occlusion) of a stented coronary artery due to thrombus formation, which may result in life-threatening complications. Moreover, the use of a stent may cause bleeding, an allergic reaction to X-ray contrast agents used to visualize the coronary arteries, and/or myocardial infarction. Accordingly, recent advances have moved away from using stent implantation toward using vascular grafts.

A vascular graft is a surgical procedure performed to redirect blood flow from one area to another, to bypass an artery that is obstructed by atherosclerosis, and/or to serve as an access point to the circulatory system for hemodialysis. A vascular graft may utilize one's own vein (autograft), expanded polytetrafluoroethylene (“ePTFE”), polyethylene terephthalate, or a different person's vein (allograft). More than 75,000 new hemodialysis grafts are placed in the U.S. each year and costs for creating and maintaining these grafts exceed $1 billion annually. Additionally, nearly 75% of arteriovenous access grafts and 20% of peripheral arterial bypass grafts will fail or become dysfunctional each year, resulting in considerable patient morbidity and substantial costs to the healthcare system. Cylerus has made considerable advancements with ePTFE grafts by using a microporous membrane for uniform fluid infusion, which has resulted in prolonging the patency of ePTFE vascular graft access (see, e.g., U.S. Pat. No. 8,721,711 and U.S. Pat. No. 8,808,255, which are described in further detail below).

U.S. Pat. No. 8,721,711 to Stephen R. Hanson, which is incorporated by reference herein in its entirety, discloses an improvement of a graft (e.g., an ePTFE graft) and an associated pump for the local delivery of a substance, e.g., a solution of sirolimus, to a patient having a hemodialysis graft. For instance, the substance may be delivered without producing unwanted systemic side effects. Such a device and system, or a similar device and system, may be utilized to deliver the novel therapeutic formulation to a patient. U.S. Pat. No. 8,808,255 discloses a drug delivery cuff that includes a drug reservoir and an integrated drug pump to be placed around any suitable vascular graft (e.g., ePTFE) or directly around a natural tissue conduit (e.g., perivascularly). Hanson describes the infusion of pharmacological agents, such as olimus, via a drug delivery cuff attached to an improved ePTFE graft. Currently, olimus is available for treatment in an oral tablet.

Effectiveness of hemodialysis treatments that utilize an ePTFE graft with a drug delivery cuff may be increased by providing a therapeutic formulation as described herein for infusion to such patients. For instance, providing the therapeutic formulation to the patients in accordance with any one or more of the techniques described herein may mitigate graft-related neointimal hyperplasia, thrombosis, and/or other processes that are capable of leading to graft failure. In one example embodiment, the therapeutic formulation may include sirolimus and/or other olimus drug(s). The therapeutic formulation may be infusible and stable in solution for a relatively long duration of time for such a pharmaceutical and therapeutic purpose.

The drug compound sirolimus, also known as RAPAMUNE® or rapamycin, has been understood to be useful in immunosuppression treatment for organ transplants and treatment of blood vessel tissue for many years. The drug and its relatives (including everolimus, zotarolimus, tacrolimus, biolimus, and others) have been used in combination with cardiovascular stents in an effort to mitigate restenosis in association with products such as the CYPHER® stent, the XIENCE® stent, the TAXUS® stent, and others.

The chemical name for sirolimus is (3S,6R,7E,9R,10R,12R,14S,15E,17E,19E,21S,23S,26R,27R,34aS)-9,10,12,13,14,21,22,23,24,25,26,27,32,33,34,34a-hexadeca-hydro-9,27-dihydroxy-3-[(1R)-2-[(1 S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacycloh-entriacontine-1,5,11,28,29(4H,6H,31H)-pentone. The molecular formula thereof is C₅₁H₇₉NO₁₃ and the molecular weight is 914.2. The chemical structure of sirolimus is as follows:

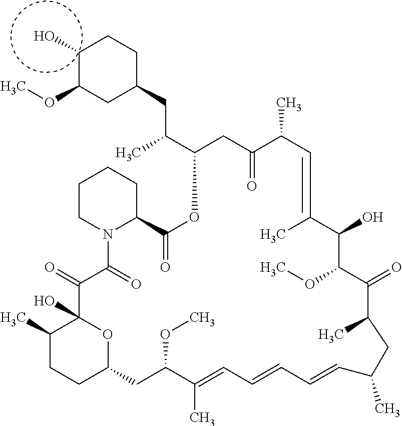 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

The related pharmaceutical “olimus” molecules are relatively similar in chemical structure, behavior, and therapeutic effect. The related molecules typically differ by replacing the hydroxyl group (circled in dashed line in the image above) with other “R” groups.

Sirolimus and its relatives are desirable compounds for use in the treatment and maintenance of hemodialysis grafts due to their therapeutic effect for inhibiting excessive tissue ingrowth, which can restrict blood flow and trigger blood clotting and thrombosis within blood vessels (neointimal hyperplasia). However, sirolimus and its relatives are virtually insoluble (e.g., substantially hydrophobic) and are chemically unstable molecules. Traditionally, no commercial parenteral formulation for sirolimus or its relatives has been available for use because the compound has not been sufficiently soluble in commonly acceptable excipients to be therapeutically significant. The oral solution of sirolimus, called RAPAMUNE® (manufactured and sold by Pfizer Inc.), contains an excipient that has not been available for use in parenteral products.

Sirolimus commonly is in the form of a crystalline powder and, as described above, is substantially insoluble in water, but may be freely soluble in benzyl alcohol, chloroform, acetone, and/or acetonitrile. Sirolimus is characterized by a low solubility in water (only 2.6 μg/ml); therefore, about 0.65 mg of sirolimus typically is dissolved in a volume of 250 ml of gastrointestinal fluid, which may not be enough to cause a therapeutic effect. In an effort to ensure that a substantial proportion (e.g., all) of the sirolimus from the oral dosage form is dissolved in the gastrointestinal fluid, sirolimus is available as an oral solution containing 1 mg/ml of sirolimus and as a tablet containing 1 mg or 2 mg of nano-sized (less than 400 nm) particles of sirolimus. However, preparation of an oral solution of sirolimus before administration typically requires a special procedure and is thus often less preferable from a patient's point of view. Sirolimus in the form of a solid dispersion is described in International Publication No. WO 97/03654. Preparation of tablets containing nano-sized particles of sirolimus is described in U.S. Pat. No. 5,989,591. The preparation of nano-sized particles of sirolimus and the preparation of sirolimus tablets containing nano-sized particles of sirolimus are both relatively complex procedures and may result in batch-to-batch variations in the dissolution of sirolimus from the tablets. Therefore, an alternative approach for the enhancement of sirolimus solubility may be desirable.

Transformation of the crystalline form of a low solubility drug to the amorphous form can significantly increase the solubility thereof, which is also true for sirolimus. However, amorphous sirolimus is chemically relatively unstable and is therefore not easily acceptable for incorporation into an oral pharmaceutical dosage form. Pharmaceutical dosage forms comprising amorphous sirolimus are described in International Publication No. WO 06/039237 and International Publication No. WO 06/094507. In WO 06/094507, a modified release pharmaceutical formulation comprising sirolimus and glyceryl monostearate at a concentration of 49.25% is described. The release rate of sirolimus from the delayed release rate formulations disclosed in WO 06/094507 is significantly suppressed compared to the marketed sirolimus formulation (RAPAMUNE®).

Marketed therapeutic agents typically require excipient(s). An excipient is a substance formulated alongside an active ingredient of a medication to provide benefit(s), such as long-term stabilization of the active ingredient, bulking up a solid formulation that includes potent active ingredient(s), and/or conferring a therapeutic enhancement on the active ingredient in the final dosage form (e.g., facilitating drug absorption, reducing viscosity, and/or enhancing solubility). An excipient may be useful in the manufacturing process to aid in the handling of an active substance, for example, by facilitating powder flowability or non-stick properties. Use of an excipient may increase in vitro stability, for example, by inhibiting (e.g., preventing) denaturation and/or aggregation over the expected shelf life of an active ingredient.

Example embodiments described herein relate to novel therapeutic formulation for use with a therapeutic agent, methods of using the formulation, and methods of making the formulation. The formulation may increase (e.g., exponentially increase) solubility and induce in vitro stability at 4° C. and 25° C. for a relatively long duration of time (e.g., at least 1 month, at least 6 months, at least 1 year, etc.), which may be significant for long-term storage. The therapeutic formulation may increase in vivo body temperature stability, which may increase treatment protocol options exponentially. Unexpectedly, the therapeutic formulation induces in vivo stability at 37° C., which is significant for delivery of therapeutic agents to a patient for effective treatment for a relatively long duration of time (e.g., up to 14 days, up to 1 month, up to 6 months, up to 1 year, etc.).

The therapeutic formulation may be especially useful for agents with notoriously low to nil solubility in water or other commonly used solvents. For example, the therapeutic formulation may be especially useful for delivery of the therapeutic agent olimus. The therapeutic formulation may be used with other therapeutic drug agents, including but not limited to actimune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cab azitaxel, emtansine, pembrolizumab, carfilzomib, nivolumab, pertuzumab, rituximab, paclitaxel, docetaxel, temsirolimus, bedamustine, panitumumab, bortezomib, venofer, and zoledronic acid.

In an example embodiment, the therapeutic formulation has a pharmaceutically acceptable shelf life such that the pharmaceutical agent, such as sirolimus, remains chemically stable (e.g., does not precipitate) for a suitable number of days (e.g., at least 10 days, at least 30 days, at least 60 days, at least 90 days, at least 180 days, at least 365 days, etc.,) at 4° C., at 25° C., and/or at 37° C. In another example embodiment, the therapeutic formulation is suitable for application to a patient's blood vessel via a delivery or infusion pump, which may be a desirable method of delivering the therapeutic formulation to a dialysis patient's hemodialysis graft location. For instance, the therapeutic formulation may be applied to a patient's blood vessel via a delivery pump, infusing continuously or intermittently, for at least 3 days, at least 7 days, at least 14 days, at least 30 days, at least 60 days, at least 90 days, at least 180 days, at least 365 days, etc.

In an example embodiment, the therapeutic formulation may be delivered via an infusion pump, which is a medical device used to deliver fluids into a patient's body in a controlled manner. Examples of an infusion pump include but are not limited to an enteral pump, a Patient-controlled analgesia (PCA) pump, an insulin pump, a continuous infusion pump, an intermittent infusion pump, a syringe pump, an elastomeric pump, a peristaltic pump, a parenteral pump, a multi-channel pump, and a “smart” pump. The infusion pump may be capable of delivering fluids in large or small amounts, and may be used to deliver the therapeutic formulation in any of a variety of ways. For instance, the infusion pump may deliver the therapeutic formulation by itself or with a treatment. Such a treatment may include therapeutic agent(s) (e.g., olimus), other medication(s) (e.g., chemotherapy drug(s), pain reliever(s), and/or antibiotic(s)), nutrient(s), and/or hormone(s) (e.g., insulin).

The therapeutic formulation may include an infusible solution that includes any suitable concentration of sirolimus (e.g., up to about 15 mg/mL of sirolimus, up to about 10 mg/mL of sirolimus, up to about 4 mg/mL of sirolimus, up to about 2 mg/mL of sirolimus, up to about 0.7 mg/mL of sirolimus, up to about 0.5 mg/mL of sirolimus, or up to about 0.2 mg/mL of sirolimus). The therapeutic formulation may satisfy any of a variety of objectives. For instance, the therapeutic formulation may be a parenteral infusible solution that can be made with pharmaceutically acceptable excipients. The therapeutic formulation may be in solution up to 0.25 mg/mL, up to 5 mg/mL, up to 50 mg/mL, etc. The infusible solution may not precipitate on infusion or injection or dilution. The infusible solution may be chemically stable for at least 3 days, for at least 30 days, for at least 6 months, or other suitable time period at 4° C., at 25° C. and/or at 37° C. The infusible solution may be compatible with infusion pumps, such as the preferred ALZET® infusion pumps. In an example embodiment, the therapeutic formulation meets all of these objectives, which is surprising and unexpected because of the compound sirolimus's insolubility and chemical degradation.

The therapeutic formulation may include a solvent that includes any one or more of the following: DSMO, ethanol, methanol, N,Ndimethylacelamide (DMA), Tween 80, polyethylene glycol (PEG) 400, castor oil, propylene glycol, glycerine, polysorbate 80, benzyl alcohol, glycerol formal, ethoxy diglycol (Transcutol, Gattefosse), tryethylene glycol dimethyl ether (Triglyme), dimethyl isosorbide (DMI), gamma-butyrolactone, N-Methyl-2-pyrrolidinone (NMP, PHARMASOLVE™), polyethylene glycol (PEG) 300 or 400, and/or polyglycolated capryl glyceride (Labrasol, Gattefosse).

For instance, a sirolimus formulation may be an FDA acceptable solvent or a combination of solvents and excipients. Examples of a solvent and/or co-solvent that may be utilized include but are not limited to DMSO, ethanol, methanol, a combination of N,N-dimethylacetamide, Tween 80, and polyethylene glycol 400, castor oil, propylene glycol, glycerine, polysorbate 80, benzyl alcohol, dimethyl formamide (DMF), glycerol formal, ethoxy diglycol (Transcutol, Gattefosse), tryethylene glycol dimethyl ether (Triglyme), dimethyl isosorbide (DMI), gamma-butyrolactone, N-Methyl-2-pyrrolidinone (NMP), polyethylene glycol 400, and/or polyglycolated capryl glyceride (Labrasol, Gattefosse).

In an example embodiment, a therapeutic drug agent (e.g., olimus) is dissolved in a therapeutic formulation that includes a combination of suitable co-solvents. Table 1 below discloses some example combinations of ethanol, polysorbate 80, PEG 300, and propylene. Each value listed for a co-solvent in Table 1 represents a percentage of the co-solvent by volume in the corresponding combination of co-solvents.

TABLE 1

| | | | | Propylene |
|---|---|---|---|---|
| | Ethanol | Polysorbate 80 | PEG-300 | Glycol |
| Combination #1 | 10 | 15 | 55 | 20 |
| Combination #2 | 15 | 20 | 50 | 15 |
| Combination #3 | 20 | 25 | 45 | 10 |
| Combination #4 | 25 | 10 | 40 | 25 |
| Combination #5 | 30 | 20 | 40 | 10 |
| Combination #6 | 25 | 20 | 45 | 10 |
| Combination #7 | 20 | 15 | 50 | 15 |
| Combination #8 | 15 | 10 | 55 | 20 |
| Combination #9 | 10 | 10 | 60 | 20 |

For instance, combination #1 includes 10% ethanol, 15% Polysorbate 80, 55% PEG-300, and 20% propylene glycol (by volume); combination #2 includes 15% ethanol, 20% Polysorbate 80, 50% PEG-300, and 15% propylene glycol; and so on.

The combinations shown in Table 1 are provided for illustrative purposes and are not intended to be limiting. It will be recognized that the therapeutic formulation may include any suitable combinations of the co-solvents. Moreover, the therapeutic formulation may include one or more co-solvents in addition to those shown in Table 1.

It will be recognized that the co-solvents listed in Table 1 may be combined in any suitable percentages. It will be further recognized that the therapeutic formulation may include co-solvent(s) in addition to those listed in Table 1. The selection of appropriate co-solvents may depend on any of variety of factors, including but not limited to the long-term stability of the therapeutic agent (e.g., olimus), the route of administration, the dosage form, and the active ingredient. For example, the combination of co-solvent may be selected for stability at 4° C., at body temperature, and/or at room temperature for at least a designated period of time (e.g., at least one month, at least three months, at least six months, or at least one year). In an example implementation, the therapeutic formulation may be delivered via an expanded polytetrafluoroethylene (ePTFE) based graft into a natural tissue conduit, e.g., a blood vessel. For example, a suitable combination may be selected for delivery into a patient via a cuff, as described by U.S. Pat. No. 8,808,255, with a microporous membrane, as described by U.S. Pat. No. 8,721,711, for uniformity of drug delivery.

In another example, a combination of co-solvents may be selected for a body temperature stable injectable olimus formulation that can be loaded into a pump connected by catheter to an ePTFE graft via a cuff to maintain function of the graft. For instance, the excipient combination may be chemically stable at 4° C., at body temperature, and/or at room temperature for at least a designated period of time (e.g., at least one month, at least three months, at least six months, or at least one year).

In another example embodiment, the therapeutic formulation is included in a solution that includes a combination of therapeutic agents. Examples of a therapeutic agent that may be included in the combination include but are not limited to olimus (e.g., sirolimus), paclitaxel, thiazolidinediones, glipizide, glimepiride, metformin, victoza, and jardiance. In another example embodiment, the therapeutic formulation may be included in a solution that includes therapeutic agent(s) to administer antibiotic(s) and/or to treat diabetes, arthritis, cancer, dehydration, migraines, pain, etc.

### Example 1

A sirolimus formulation has been described for use with ALZET® osmotic minipumps (based on a combination of N,N-dimethylacetamide, Tween 80, and polyethylene glycol 400) that is stable for about 2 weeks at 37° C. (109). Other methods that may be used to solubilize rapamycin are described by P. Simamora et al. 2001. Solubilization of Rapamycin. Int J Pharm 213 (1-2):25-9.

The following three candidate formulations were selected for stability testing:
(1) 10% DMA/20% polysorbate 80/50% PEG 300/20% propylene glycol;
(2) 15% absolute ethanol/20% polysorbate 80/50% PEG 300/15% propylene glycol;
(3) 12.5% PHARMASOLVE™/20% polysorbate 80/50% PEG 300/17.5% propylene glycol.

These candidate formulations were first tested for compatibility with a drug delivery system (here, the ALZET® Osmotic Pump Model 2ML4) that was suitable for use in animal studies. All three candidate formulations were found to be compatible using the ALZAID® Chemical Compatibility Test Kit.

For stability testing, sirolimus was initially formulated to a solution strength of 700 μg/mL which was thought to approximate the solution strength that would be required in animals implanted with osmotic pumps while simultaneously avoiding systemic exposure above 1 ng/mL, which is understood to be potentially immunosuppressive. Subsequent animal studies included testing using sirolimus solution strengths of 285 μg/mL and 855 μg/mL, based on expected animal body weights, pump reservoir volume and flow rates.

Samples of the candidate formulations were loaded into osmotic minipumps in duplicate, connected to polyethylene catheters and submerged in saline and held at 37° C. in a waterbath. Samples for stability analysis were collected at time 0, days 7, 14, 21 and 30 and were submitted to an analytical lab (PharmOptima, Kalamazoo, Mich.) for determination of sirolimus concentration.

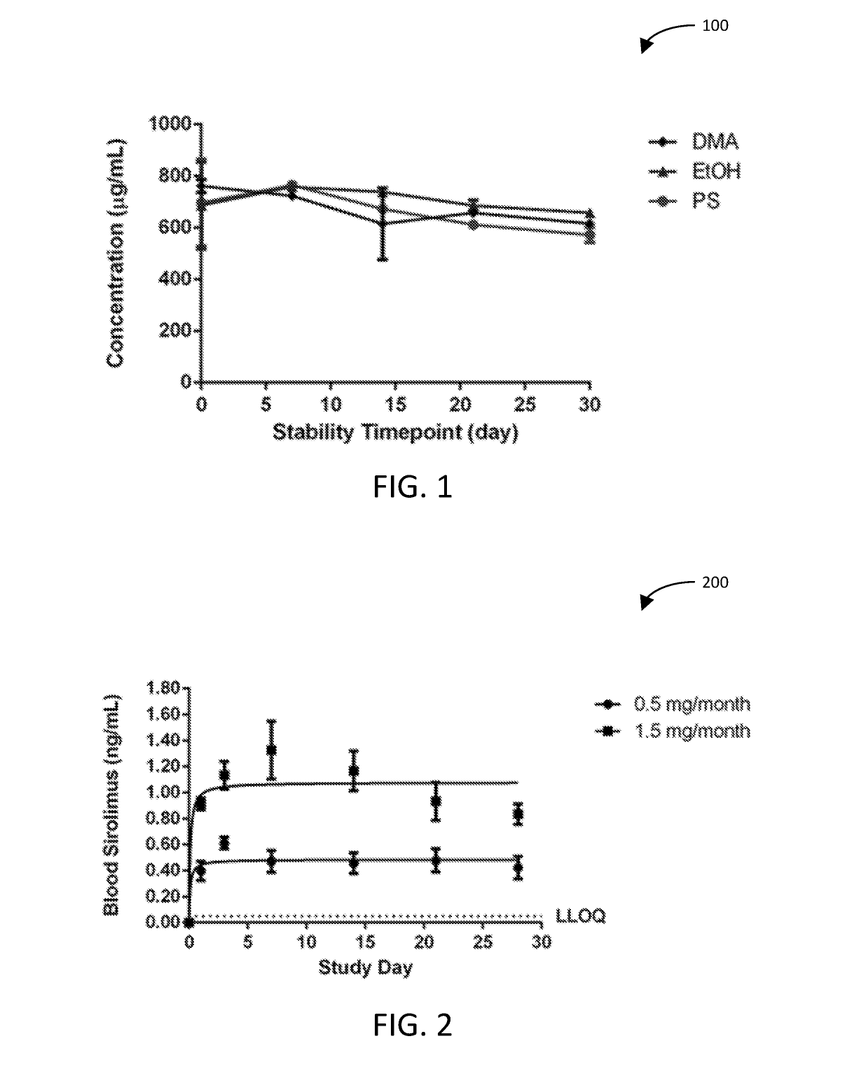

FIG. 1 depicts a graph 100 showing stability of three example formulations that include sirolimus in accordance with an embodiment. The three example formulations include a dimethylacetamide-based (DMA-based) formulation as defined above, an ethanol-based (EtOH-based) formulation, and a Pharmasolve-based (PS-based) formulation. Data in the graph reflect mean of two replicate minipumps+/−standard deviation. As shown in FIG. 1, all three example formulations exhibited relatively high stability with the ethanol-based formulation being numerically superior to the other two. At 30 days, the ethanol-based formulation had retained 94.0% of its nominal concentration (700 μg/mL) and 95.8% of its time-zero sample concentration of 687 μg/mL. By contrast, the DMA-based formulation retained 88.0% and 80.8% (nominal and time-zero) sirolimus concentration at day 30. The PS-based formulation retained 81.7% and 82.1% (nominal and time-zero) sirolimus concentration at day 30.

Based on the aforementioned results, the ethanol-based formulation was selected for follow-up animal studies.

### Example 2

While ethanol formulation is adequate for the animal study conducted in follow-up, for human applications the sirolimus concentration required is likely to be higher. It is believed that a 3 month delivery in humans will greatly prolong the time to graft failure, which supports using a DUROS® pump (DURECT® Corporation) with a 3-month delivery duration.

DUROS® pumps are the functional and clinically approved for human use equivalents of the ALZET® minipumps used for the follow-up animal research. In an example, sirolimus was formulated to 3 mg/mL, roughly 4-times the concentration being used in the follow-up animal studies, for use with a DUROS® pump. The stability of the above-identified (EXAMPLE 1) ethanol based formulation with the sirolimus concentration of 3.0 mg/mL was tested. After 30 days at 37° C., the formulation was assayed to be 2.52 mg/mL, approximately 83% of starting solution strength.

A formulation of sirolimus with 30 day stability at 37° C. was identified that is suitable for animal studies. The formulation uses solvents and excipients that are likely FDA-approvable based on precedents. The stability of a sirolimus formulation at a concentration (3 mg/mL) that would support the above-identified intended clinical use was also demonstrated.

In a pilot study performed by Dr. Stephen R. Hanson, baboons (15-18 kg) were implanted with ALZET® minipumps that delivered sirolimus at a rate of about 2 mg/month. These infusion rates achieved blood Cₘₐₓ levels of 1.5-6 ng/mL in the first week (exceeding the desired 1-2 ng/mL threshold for sirolimus-induced immunosuppression) and blood levels of sirolimus that declined steadily thereafter, presumably as a result of formulation instability. Through application of a variety of scaling methods, delivery of 0.5 and 1.5 mg/month in ˜60 kg pigs was selected as a means of delivering steady state sirolimus at a level that approached the 1-2 ng/mL exposure limit.

Accordingly, sirolimus was prepared in the above-identified ethanol formulation at solution strengths of 285 and 855 μg/mL and preloaded into ALZET® osmotic minipumps (model 2ML4), which deliver their loaded volume of 2 mL over a 4 week period. The minipumps were loaded in a sterile field and osmotically primed by submersion in sterile saline at 37° C. for a period of at least 48-hours prior to implantation.

Six pigs (55-65 kg) were anesthetized with telazol (5 mg/kg) combined with ketamine (5 mg/kg) and xylazine (1 mg/kg) given IM. The pigs were intubated and maintained on oxygen and isoflurane inhalation anesthesia. The pigs were outfitted for routine physiologic monitoring. A small incision was made in the ventral surface of the neck to implant a minipump with its catheter introduced at the external jugular and advanced into the vena cava. Incisions were closed and animals recovered from anesthesia. Blood samples were drawn into tubes containing K₂-EDTA at the time of implant (day 0) and via an ear vein at 1, 3, 7, 14, 21, and 28 days. At 28 days the animals were terminated. Whole blood samples were stored frozen until shipped to PHARMOPTIMA LLC for sirolimus assay.

FIG. 2 depicts a graph 200 showing whole blood levels of sirolimus in pigs infused intravenously (through external jugular vein) with 0.5 or 1.5 mg/month stable sirolimus formulations for 28 days via osmotic ALZET® minipumps placed subcutaneously in accordance with an embodiment. As shown in FIG. 2, mean sirolimus levels for the pigs on day 0 were beneath the lower limit of quantitation (LLOQ) of 50 pg/mL for the whole blood assay. In the early phase of the experiment, sirolimus levels reached systemic peak levels (Cₘₐₓ) of 0.611±0.045 ng/mL and 1.327±0.223 ng/mL (mean±std dev) in the 0.5 and 1.5 mg/month groups (n=3 each), respectively. These peak exposures occurred at (Tₘₐₓ) day 3 and 7 of the 0.5 and 1.5 mg/month groups, respectively.

Following the observed peak in both groups and at all time points beyond time zero, there was an approximate 2-3 fold separation in whole blood sirolimus between the low dose (0.5 mg/month) and high dose (1.5 mg/month) cohorts as would be expected from a 3-fold separation in the delivered dose.

The apparent decline from peak to day 28 sample of 31-37% was observed in both cohorts. This may be related to an artifact peak arising from early performance of ALZET® minpumps and their initial flow rates where viscous solutions are used, some loss of sirolimus formulation API in the drug reservoir during the 28-day study, or a mixed contribution of both.

Blood levels of sirolimus were fitted to a one-phase exponential function (R²=0.812). Model parameters indicate that steady-state levels of sirolimus were achieved at 0.487 ng/mL (0.439-0.535; 95% CI) and 1.078 ng/mL (0.971-1.186; 95% CI) in the 0.5 and 1.5 mg/month groups, respectively.

The selected doses of sirolimus and the delivery mechanism capable of delivering a high local exposure of sirolimus at the infusion site were selected to limit systemic exposure to no more than 1-2 ng/mL. The testing in ˜60-kg pigs achieved 28-days continuous dosing of sirolimus and a steady-state blood level of 0.487 and 1.078 ng/mL based on model fitting the data.

### Example 3

In an example embodiment, the therapeutic formulation includes 15% absolute ethanol, 20% polysorbate 80, 50% PEG 300, and 15% propylene glycol. When applied, solution strength may be 878 μg/g.

The plan to evaluate 4° C. storage stability of such a formulation was conducted according to the following time/testing scheme:

TABLE 2

| STABILITY TIME | TUBE ID |
|---|---|
| time 0 | t0 A/B |
| 1 week | 1 wk A/B |
| 2 week | 2 wk A/B |
| 1 month | 1 mo A/B |
| 2 month | 2 mo A/B |
| 4 month | 4 mo A/B |
| 6 month | 6 mo A/B |

The above-referenced formulation was manufactured and was frozen. The formulation was thawed for osmotic pump fill a week later. The formulation was transferred to 14×200 μl aliquots in cryo tubes the following day for a stability study. These aliquots were stored in a refrigerator (at 4° C.) for times specified above in Table 2. Following the experimental portion of the stability study, the aliquots were transferred to deep freeze (−20° C.) and held frozen until analysis.

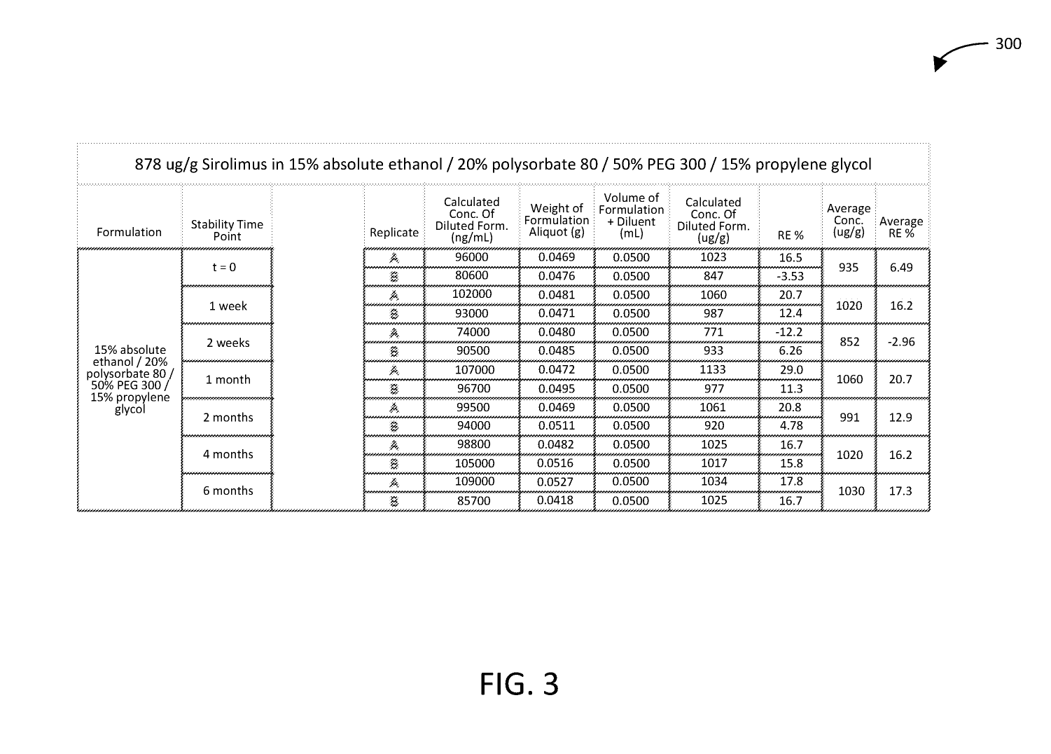

FIG. 3 depicts a table 300 that shows detailed results of the stability study in accordance with an embodiment. As shown in FIG. 3, the stability study established that an example embodiment demonstrated 4° C. stability of the therapeutic solution including sirolimus for at least 6 months. Accordingly, at least some example embodiments are capable of achieving 4° C. stability for relatively long durations of time (e.g., at least 6 months).

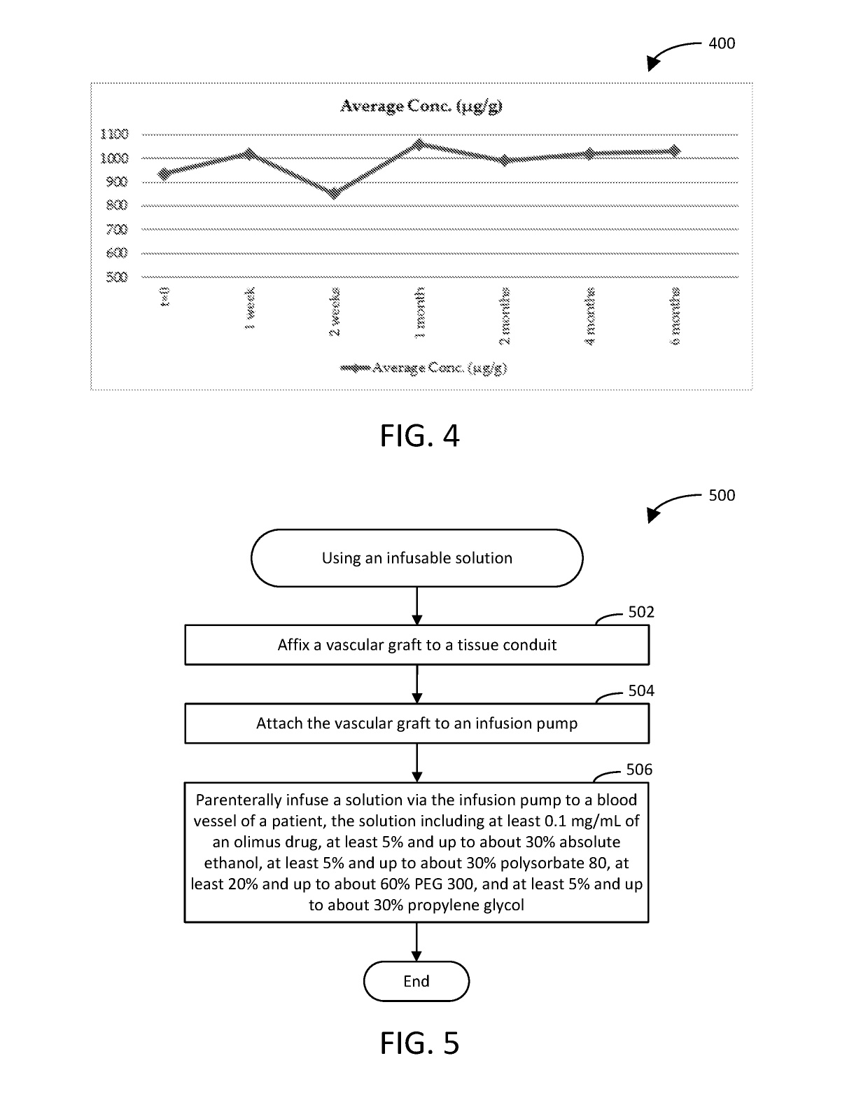

FIG. 4 depicts a graph 400 that shows summary results of the stability study in accordance with an embodiment. The measured average concentration of sirolimus at each timepoint (second to last column of Table 2) is plotted across time in FIG. 4. Over a 6-month period of time, sirolimus is measured to between 97% and 120% of the starting value for sirolimus concentration measured at time 0.

As illustrated in FIG. 4, the average concentration of the samples studied over the 6-month course were maintained. This establishes that the formulation in the example embodiment is pharmaceutically acceptable and compatible in terms of stability as a refrigerated product.

FIG. 5 depicts a flowchart 500 of an example method for using an infusible solution in accordance with an embodiment. As shown in FIG. 5, the flowchart starts at step 502. In step 502, a vascular graft is affixed to a tissue conduit. At step 504, the vascular graft is attached to an infusion pump. At step 506, a solution is parenterally infused via the infusion pump to a blood vessel of a patient. The solution includes at least 0.1 mg/mL of an olimus drug, at least 5% and up to about 30% absolute ethanol, at least 5% and up to about 30% polysorbate 80, at least 20% and up to about 60% PEG 300, and at least 5% and up to about 30% propylene glycol.

In an example embodiment, step 506 includes causing a therapeutic concentration of the olimus drug to be maintained in a local tissue to which the olimus drug is infused for at least a designated number of days. For instance, the designated number of days may be 3 days, 5 days, 14 days, 30 days, or 60 days. It will be recognized by persons skilled in the art that the solution is described as being parenterally infused via the infusion pump for illustrative purposes and is not intended to be limiting. For instance, an infusion pump need not necessarily be utilized to parenterally infuse the solution.

In one example embodiment, the solution includes up to about 3.0 mg/mL of an olimus drug, at least 10% and up to about 20% absolute ethanol, at least 15% and up to about 25% polysorbate 80, at least 45% and up to about 55% PEG 300, and at least 10% and up to about 20% propylene glycol. In another example embodiment, the solution includes up to about 3.0 mg/mL of an olimus drug, about 15% absolute ethanol, about 20% polysorbate 80, about 50% PEG 300, and about 15% propylene glycol. The olimus drug may include sirolimus, everolimus, zotarolimus, tacrolimus, and/or biolimus, though the scope of the example embodiments is not limited in this respect.

In an example embodiment, the solution is configured to cause the olimus drug to not precipitate on infusion, injection, and/or dilution. In another example embodiment, the solution is configured to cause the olimus drug to not precipitate for at least a designated number of days at a designated temperature. For instance, the designated number of days may be 30 days, 60 days, 90 days, one year, etc. The designated temperature may be 4° C., 25° C., or 37° C. In yet another example embodiment, the vascular graft is an expanded polytetrafluorethylene (ePTFE) graft connected to a drug delivery cuff.

It will be recognized that the solution may include any suitable therapeutic agent and any suitable solvents. It will be further recognized that the therapeutic agent may be provided in any suitable concentration in the solution, and the solvents may be provided in any suitable proportions in the solution. In some example embodiments, one or more steps 502, 504, and/or 506 of flowchart 500 may not be performed. Moreover, steps in addition to or in lieu of steps 502, 504, and/or 506 may be performed.

### III. Conclusion

Various embodiments of the invention have been described above. Although this invention has been described with reference to these specific embodiments, the descriptions are provided for illustrative purposes and are not intended to be limiting. Those skilled in the art will recognize, or be able to ascertain using no more than routine experimentation, many equivalents to the specific embodiments of the invention described herein. Such equivalents are intended to be encompassed by the following claims. For instance, various modifications and applications may occur to those skilled in the art without departing from the scope of the invention as defined by the following claims.

What is claimed is:

1. A pharmaceutical formulation, comprising:
at least 5% absolute ethanol;
at least 5% polysorbate 80;
at least 20% PEG 300; and
at least 5% propylene glycol.

2. The pharmaceutical formulation of claim 1, further comprising at least 0.5 mg/mL of an olimus drug in a solution.

3. The pharmaceutical formulation of claim 2, wherein the olimus drug includes at least one of sirolimus, everolimus, zotarolimus, tacrolimus, or biolimus.

4. The pharmaceutical formulation of claim 2, wherein the solution is a parenteral infusible solution.

5. The pharmaceutical formulation of claim 2, wherein the solution is configured to cause the olimus drug to not precipitate on at least one of infusion, injection, or dilution.

6. The pharmaceutical formulation of claim 2, wherein the solution is soluble and configured to cause the olimus drug to not precipitate for at least 10 days at one or more of the following temperatures: 4° C., 25° C., or 37° C.

7. The pharmaceutical formulation of claim 2, wherein the solution is suitable for application to a blood vessel of a patient via an infusion pump.

8. The pharmaceutical formulation of claim 1, comprising a solution of:
up to about 3.0 mg/mL of an olimus drug;
at least 10% and up to about 20% absolute ethanol;
at least 15% and up to about 25% polysorbate 80;
at least 45% and up to about 55% PEG 300; and
at least 10% and up to about 20% propylene glycol.

9. The pharmaceutical formulation of claim 1, comprising a solution of:
up to about 3.0 mg/mL of an olimus drug;
about 15% absolute ethanol;
about 20% polysorbate 80;
about 50% PEG 300; and
about 15% propylene glycol.

10. A pharmaceutical formulation, comprising:
up to about 30% absolute ethanol;
up to about 30% polysorbate 80;
up to about 60% PEG 300; and
up to about 30% propylene glycol.

11. The pharmaceutical formulation of claim 10, further comprising at least 0.5 mg/mL of an olimus drug in a solution.

12. The pharmaceutical formulation of claim 11, wherein the olimus drug is selected from the group consisting of: sirolimus, everolimus, zotarolimus, tacrolimus, and biolimus.

13. The pharmaceutical formulation of claim 10, wherein the solution is a parenteral infusible solution.

14. The pharmaceutical formulation of claim 10, wherein the solution is configured to cause the olimus drug to not precipitate on at least one of infusion, injection, or dilution.

15. The pharmaceutical formulation of claim 10, wherein the solution is configured to cause the olimus drug to not precipitate for at least 10 days at one or more of the following temperatures: 4° C., 25° C., or 37° C.

16. The pharmaceutical formulation of claim 10, wherein the solution is suitable for application to a blood vessel of a patient via an infusion pump.

17. A method of mitigating thrombosis in a blood vessel having a hemodialysis graft, the method comprising:
parenterally infusing a solution to a blood vessel of a patient, the solution comprising at least 0.1 mg/mL of an olimus drug, at least 5% and up to about 30% absolute ethanol, at least 5% and up to about 30% polysorbate 80, at least 20% and up to about 60% PEG 300, and at least 5% and up to about 30% propylene glycol.

18. The method of claim 17, wherein the olimus drug includes at least one of sirolimus, everolimus, zotarolimus, tacrolimus, or biolimus.

19. The method of claim 17, wherein parenterally infusing the solution comprises:
causing a therapeutic concentration of the olimus drug to be maintained in a local tissue to which the olimus drug is infused for at least 3 days.

20. The method of claim 17, wherein the solution is configured to cause the olimus drug to not precipitate on infusion.

21. The method of claim 17, wherein the solution is configured to cause the olimus drug to not precipitate for at least 30 days at one or more of the following temperatures: 4° C., 25° C., or 37° C.
